(19) United States
(12) Patent Application Publication
(10) Pub. No.: US 2016/0038219 A1
(43) Pub. Date: Feb. 11, 2016

(54) MULTIPURPOSE ELECTROSURGICAL DEVICE

(71) Applicant: Medtronic Advanced Energy LLC, Minneapolis, MN (US)

(72) Inventors: Jonathan J. Barry, Stratham, NH (US); Joseph Sylvester, Andover, MA (US); Nathan Zamarripa, Kittery Point, ME (US)

(21) Appl. No.: 14/456,746

(22) Filed: Aug. 11, 2014

(51) Int. Cl. A61B 18/14

(52) U.S. Cl. CPC A61B 18/1442; A61B 2018/1475

(57) ABSTRACT

An electrosurgical device and methods of use thereof. In an embodiment, the device comprises a multipurpose electrosurgical device comprising a handle, a shaft distal to the handle, a first, retractable electrode and a second non-retractable electrode, the electrodes adjacent a distal end of the shaft. The device may be selectively configured for use in a bipolar and a monopolar mode and may provide for selective extension and retraction of the retractable electrode. The retractable electrode may be selectively extended and retracted into a cavity within the shaft. When the retractable electrode is in a retracted position, the device may be configured for use in a monopolar mode. When the retractable electrode is in an extended or fully extended position, the device may be configured for use in a bipolar mode.

### FIELD

This disclosure relates generally to the field of medical devices, systems and methods for use in surgical procedures. More specifically, this disclosure relates to electrosurgical devices, systems and methods that provide for cutting, coagulation, hemostasis and sealing of bodily tissues with a single electrosurgical device.

### BACKGROUND

Historically two distinct electrosurgical devices, monopolar and bipolar, were required for performing different functions, such as tissue cutting and tissue sealing. Thus, when required to perform these different functions in a surgical procedure, physicians were required to switch between different devices which could result in longer procedure times, higher costs and more room for inaccuracies. To address this need, some electrosurgical devices capable of performing both cutting and sealing of tissue, including fluid-assisted sealing of tissue, have been developed and are described, for example, in U.S. Pat. No. 8,632,533 to Greeley, et al., U.S. Patent Application Publication No. 2012/0004657 (hereinafter, “the ‘657 Application) to Conley, et al., U.S. Patent Application Publication No. 2011/0178515 to Bloom et al, (hereinafter, “the ‘515 Application), each assigned to the assignee of the present disclosure and incorporated by reference herein in their entireties to the extent they are consistent with the present disclosure.

Many devices that have been developed include a handpiece having two electrodes which may be configured as bipolar electrodes connected to a source of bipolar power. To operate the same two-electrode device in a monopolar mode, one of the two electrodes may be selectively deactivated and the other of the two electrodes coupled to a source of monopolar power. As known, during monopolar operation, the monopolar electrode of the device may be used in conjunction with a ground pad dispersive electrode placed on a patient (also known as a patient return electrode or neutral electrode). In this manner, the dual function device may provide treatment to tissue utilizing one or both electrodes depending upon the desired tissue treatment. Despite having the ability to perform different functions with a single device, when monopolar function is desired, for example to cut tissue, only one of the two electrodes of the device are utilized and the second (deactivated, unused during monopolar operation) electrode may pose an obstruction to the field of view of a user of the device during the monopolar operation. Furthermore, the unused electrode may unnecessarily prevent the monopolar electrode from entering smaller spaces or tissue areas that could otherwise be accessed if the unused electrode was not exposed. Therefore, it would be beneficial to provide a device capable of performing multiple functions and having aselectively moveable deactivated or unused electrode so as to selectively move the deactivated or unused electrode from the surgical field of view or working electrode area during an electrosurgical procedure.

### BRIEF DESCRIPTION OF THE DRAWINGS

FIG. 1A is a front view of an embodiment of a system according to the present disclosure including an electrosurgical unit in combination with a fluid source and handheld electrosurgical device;

FIG. 1B is a front view of an embodiment of a system according to the present disclosure including an electrosurgical unit having a cartridge receptacle, the electrosurgical unit in combination with a fluid source and a handheld electrosurgical device;

FIG. 2 is a partial side view of an electrosurgical device with a retractable electrode in a fully extended position according to an embodiment of the present disclosure;

FIG. 3 is a partial side view of an electrosurgical device with a retractable electrode in a retracted position according to an embodiment of the present disclosure;

FIG. 4A is a close-up perspective view of a first side of first and second electrodes of an electrosurgical device according to an embodiment of the present disclosure.

FIG. 4B is a close-up perspective view of a second side of the first and second electrodes of an electrosurgical device according to an embodiment of the present disclosure.

FIG. 5 is a close-up cross-sectional view of a distal portion of an electrosurgical device showing a retractable electrode in a retracted position, a second electrode and a fluid flow path according to an embodiment of the disclosure.

FIG. 6 is a side cross-sectional view of an electrosurgical device coupled to a cartridge assembly according to an embodiment of the disclosure.

FIG. 7 is a close-up side cross-sectional view of a handle of an electrosurgical device, according to an embodiment of the disclosure.

### DETAILED DESCRIPTION

Throughout the description, like reference numerals and letters indicate corresponding structure throughout the several views. Also, any particular features(s) of a particular exemplary embodiment may be equally applied to any other exemplary embodiment(s) of this specification as suitable. That is, features between the various exemplary embodiments described herein are interchangeable as suitable and may not be exclusive. From the specification, it should be clear that the terms “distal” and “proximal” are made in reference to a user of the device.

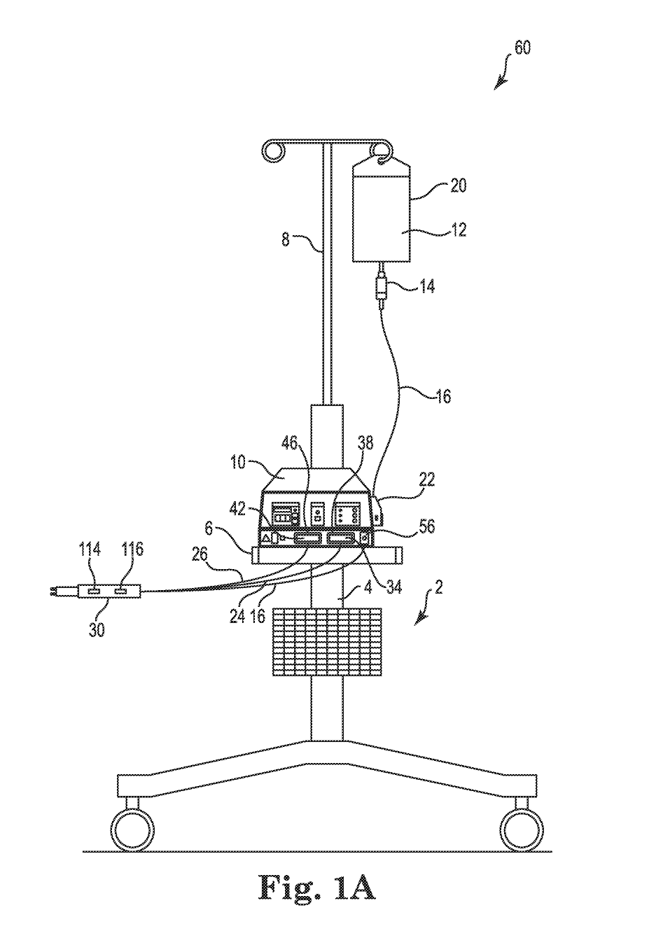

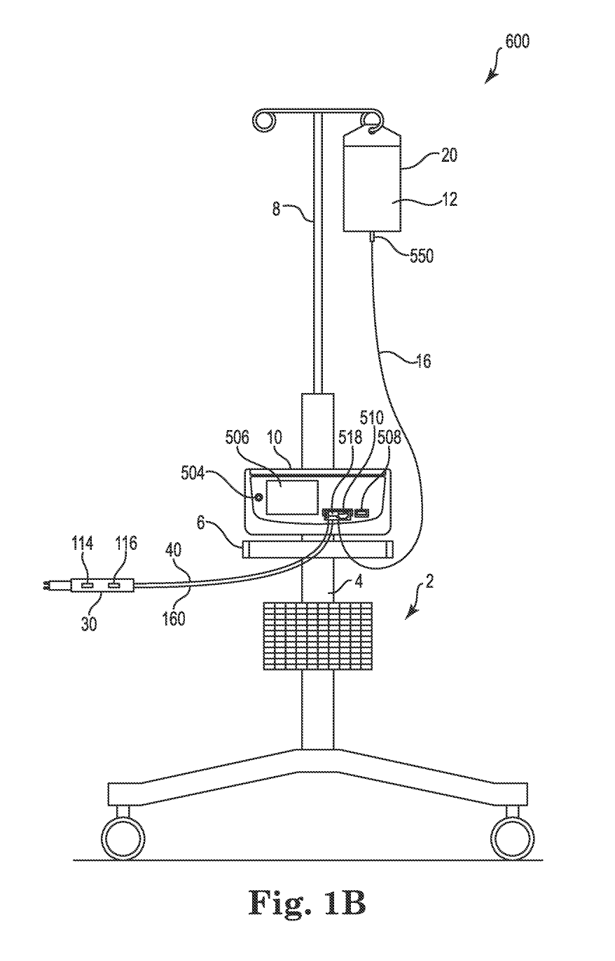

FIG. 1A shows a front view of one embodiment of a system 60 of the present disclosure which may include an electrosurgical unit 10 in combination with a fluid source 20 and a handheld electrosurgical device 30. As further described herein, the device 30 may comprise a multipurpose device, configurable for use in cutting and sealing of tissue. The device 30 may thus be configurable for use in both a monopolar and a bipolar mode. In addition, FIG. 1A shows a movable cart 2 having a support member 4 comprising a hollow cylindrical post which carries a platform 6 comprising a pedestal table to provide a flat, stable surface for location of the electrosurgical unit 10.

As shown, cart 2 further comprises a fluid source carrying pole 8 having a height which may be adjusted by sliding the carrying pole 8 up and down. Fluid source 20 can be supported at the top of pole 8. Fluid source 20 may comprise a bag of fluid from which fluid 12 may flow through a chamber 14, to delivery tubing 16 and to handheld electrosurgical device 30. The fluid delivery tubing 16 passes through pump 22.

The electrosurgical unit 10 may be configured to provide both monopolar and bipolar radio-frequency (RF) power output. However, electrosurgical unit 10 may include a lock out feature preventing both monopolar and bipolar output from being simultaneously activated. During monopolar operation of electrosurgical device 30, a first electrode, often referred to as the active electrode, may be provided with electrosurgical device 30 while a second electrode (not shown), often referred to as the indifferent or neutral electrode, may be provided in the form of a ground pad dispersive electrode located on a patient (often referred to as a patient return electrode), typically on the back or other suitable anatomical location. During bipolar operation of electrosurgical device 30, the ground pad electrode located on the patient is not required, and a second electrode providing a second electrical pole may be provided as part of the device. As indicated above, monopolar and bipolar power may be provided from electrosurgical unit 10 as known in the art, or from separate electrosurgical units. Further details of suitable embodiments of fluid flow, pumping of fluid as well as monopolar and bipolar operation of an electrosurgical unit 10 and device 30 may be such as described, for example in the '657 Application.

As shown in FIG. 1A, the electrosurgical device 30 may be connected to electrosurgical unit 10 via cables 24 and 26. Cable 24, with plug 34, connects to bipolar output receptacle 38 while cable 26, with plug 42, connects to monopolar output receptacle 46 of electrosurgical unit 10. When electrosurgical unit 10 may be used in monopolar mode, an additional cable (not shown) may connect a ground pad electrode (not shown) to a ground pad receptacle of the electrosurgical unit 10. Detailed descriptions and schematic drawings describing operation of electrosurgical unit 10 and particularly operation of electrosurgical unit 10 in conjunction with bipolar and/or monopolar electrosurgical devices are provided in the '657 Application.

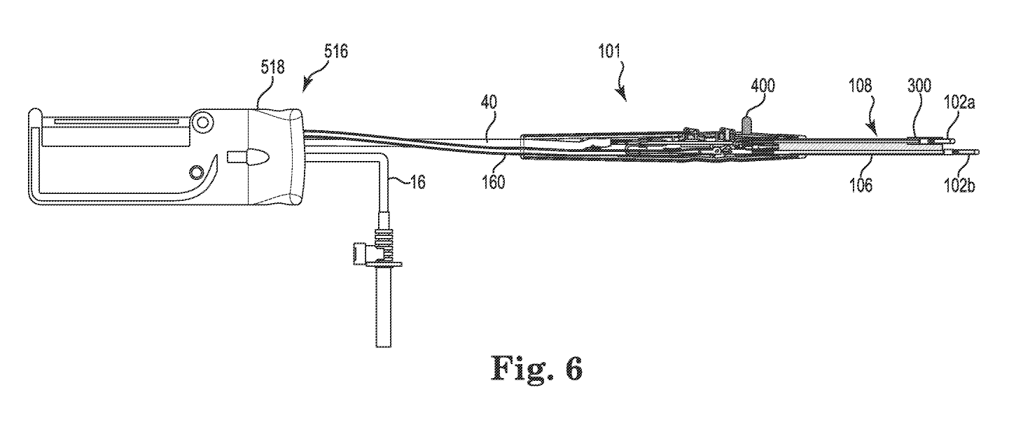

FIG. 1B shows a front view of another embodiment of a system 600 of the present disclosure which includes an electrosurgical unit 500 in combination with a fluid source 20 and a handheld electrosurgical device 30 having an electrical cable 40 and a fluid delivery tubing segment 160 extending therefrom. Electrosurgical unit 500 may be an electrosurgical unit such as electrosurgical unit 300 of the '515 application. Electrosurgical unit 500 includes a power (on/off) switch 504, a graphical user interface (GUI) 506 and a ground receptacle 508. A cartridge receptacle 510 is provided on the unit 500 for receiving a cartridge member 518. Monopolar and bipolar power may be provided from electrosurgical unit 500 in a manner known in the art. With this construction, electrosurgical device 30 may be connected to electrosurgical unit 500 via a cartridge assembly 516 comprising a cartridge member 518 (FIG. 6). Electrical cable 40 and tubing segment 160 of device 30 may be connected to a cartridge 518. The cartridge 518 and cartridge assembly 516 may be constructed in accordance with cartridge assembly 516 of the '515 Application. Detailed descriptions and schematic drawings describing operation of electrosurgical unit 500 and particularly operation of electrosurgical unit 500 in conjunction with bipolar and/or monopolar electrosurgical devices are provided in the '515 Application. With system 600, fluid 12 from fluid source 20 may be communicated through an enclosed fluid passage provided by various structures. Fluid 12 flows from the fluid source 20 into cartridge member 518 of cartridge assembly 16 (FIG. 6) through a lumen of the flexible fluid delivery tubing 16. At one end, fluid delivery tubing 16 couples to fluid source 20 after the fluid source 20 may be penetrated with a spike 550 located at the end of the tubing 16 in a known manner. Fluid delivery tubing 16 may be made of a plastic material, such as flexible polyvinyl chloride (PVC) or other flexible material such as an elastomer.

At its opposite end, fluid delivery tubing 16 may be coupled and tightly fastened with a fluid tight connection to cartridge member 18. A fluid delivery tubing segment 160 is connected to the device handle 101 (FIG. 6) at one end and to the cartridge member 18 at an opposite end. Tubing segment 160 is coupled and tightly fastened with a fluid tight connection to cartridge member 18. Coupling of fluid delivery tubing 16 to the cartridge member 18 and fluid flow from delivery tubing 16 to tubing segment 160 through cartridge member 18 may be as described, for example, in the '515 Application. Fluid flow through tubing segment 160 allows for fluid to flow through the device handle 101 and to a distal end of the device as will be described more fully herein below.

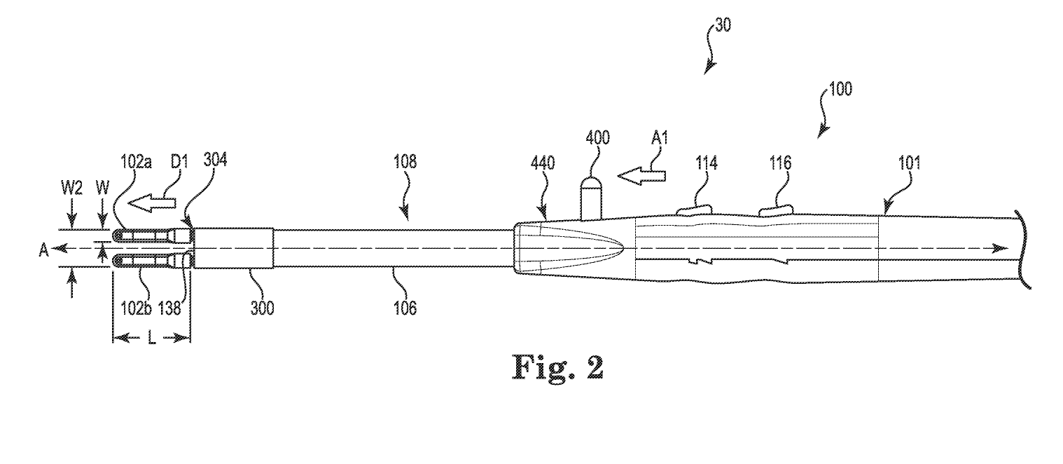

An exemplary electrosurgical device 30 of the present disclosure, which may be used in conjunction with electrosurgical unit 10 or electrosurgical unit 500 is shown in FIG. 2. While various electrosurgical devices of the present disclosure are described herein with reference to use with electrosurgical units 10, 500, it should be understood that the description of the combination is for purposes of illustrating the systems of the disclosure. Consequently, it should be understood that while the electrosurgical devices disclosed herein may be disclosed for use with electrosurgical units 10, 500, it may be plausible to use electrosurgical devices disclosed herein with another electrosurgical unit.

As shown in FIG. 2, exemplary device 30 includes an elongated handpiece 100 with a handle 101. Handpiece 100 may be configured to enable a user of device 30 to hold and manipulate device 30 between the thumb and index finger like a writing instrument. Handle 101 may comprise a sterilizable, rigid, electrically insulative material, such as a synthetic polymer (e.g., polycarbonate, acrylonitrile-butadiene-styrene).

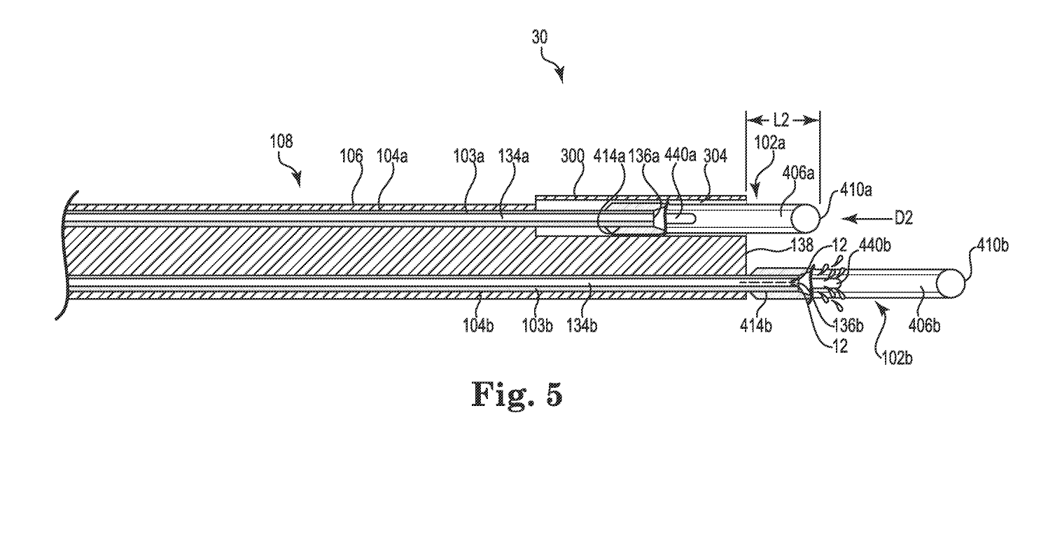

With reference between FIGS. 2 and 5, the handpiece 100 includes an elongated, rigid, electrically insulative shaft 108 comprising a shaft body 106 extending from the handle 101. The shaft body 106 may comprise a sterilizable, rigid, electrically insulative material such as a synthetic polymer. The shaft 108 may also comprise a shaft sleeve 300 provided at a distal end of the shaft 108 and shaft body 106. The handpiece 100 includes push buttons 114 and 116 which comprise hand switch assemblies (not shown) as known in the art for forming a closed circuit which may be sensed by electrosurgical units 10, 500 to then selectively provide bipolar or monopolar power respectively.

A first electrode 102a and a second electrode 102b extend from a distal end 138 of the shaft 108 and are coupled to parallel, self-supporting, electrically conductive hollow fluid delivery shafts 103a, 103b which comprise metal such as stainless steel tubing. Shafts 103a, 103b are in turn coupled to a source of electrical energy (e.g., monopolar and/or bipolar power of electrosurgical units 10, 500). Shafts 103a, 103b extend through linear conduits provided by cylindrical through passages 104a, 104b of shaft 108. Shafts 103a, 103b include through passages or fluid delivery lumens 134a, 134b and at a distal end comprise fluid outlets 136a, 136b. Fluid delivery lumens 134a, 134b allow for fluid delivery out of outlets 136a, 136b to tissue from fluid delivery tubing 16, 160 such as described above.

As can be seen in FIG. 5, shaft 108 includes a cavity 304 adjacent the one of the first and second electrodes 102a, 102b. Cavity 304 is sized to receive at least a portion of the first electrode 102a, as described more fully herein below. In the illustrated embodiment, the cavity 304 is formed by the shaft sleeve 300 and is adjacent at least the first electrode 102a. The first electrode 102a may be said to be an upper electrode, provided at the top of the handpiece 100 in relation to the orientation of the handpiece as shown in FIG. 2. When held by a user, the device may be held with push buttons 114, 116 viewable and facing a user's line of sight. In other words, push buttons 114, 116 and electrode 102a may be considered to be in an upper or top position. Thus, electrode 102a may be the first electrode viewable in the user's line of sight when both electrodes 102a, 102b are extended from the device 30, as will be further described herein. The second electrode 102b may conversely be said to be provided as a lower electrode or positioned at a bottom of the handpiece 100 (i.e., spaced circumferentially about an axis A of handle 101 approximately 180 degrees from the circumferential position of push buttons 114, 116).

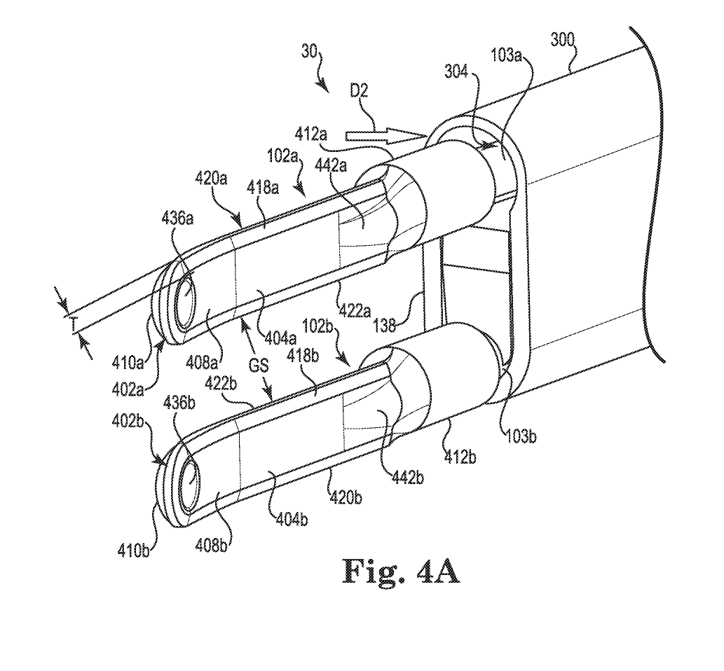

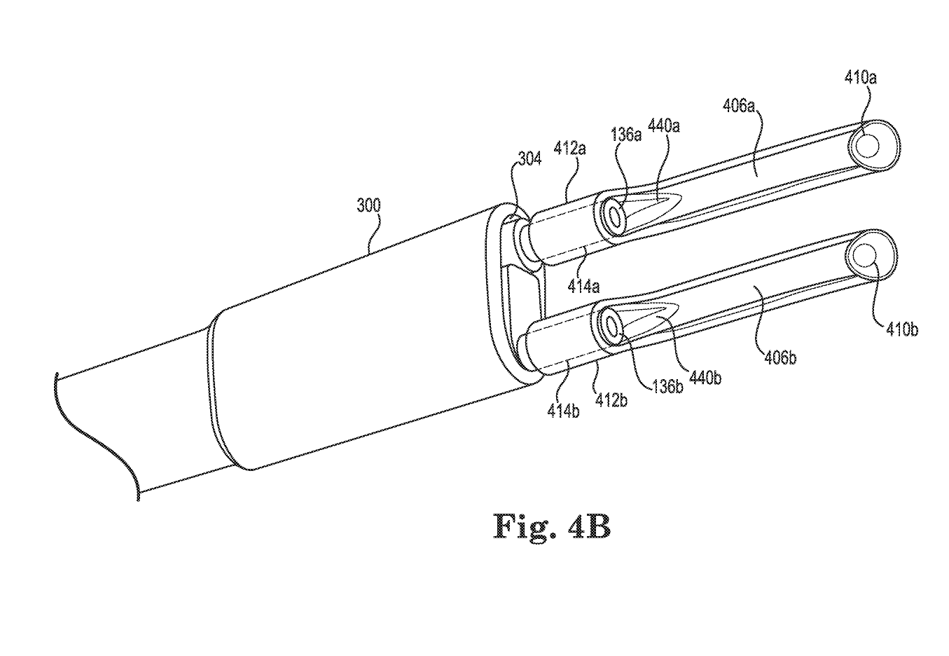

FIG. 4A provides a close-up view of a first side 404a, 404b of each of the first and second electrodes 102a, 102b and a close-up view of a distal portion of shaft sleeve 300 and cavity 304. As shown, electrodes 102a, 102b may be arranged to provide two laterally and spatially separated (by empty space) electrode tips which may be configured as mirror images in size and shape, and may have a blunt, rounded distal end which provides a smooth continuous surface (e.g., may be devoid of points or edges) to treat tissue. Electrodes 102a, 102b may also be referred to herein as “electrode tips 102a, 102b” and comprise an electrically conductive metal, such as stainless steel. Other suitable materials may include titanium, gold, silver and platinum. With respect to spacing, in the present embodiment the spatial gap separation GS between electrodes 102a, 102b may be in a range for example of any increment less than 4 mm.

Electrodes 102a, 102b each comprise opposing first (top) and second (under) sides 404a/406a, 404b/406b, (second sides 406a, 406b shown in FIG. 4B) a medial edge 422a, 422b and a lateral edge 420a, 420b. Electrodes 102a, 102b each comprise an elongated blade-shaped member with the lateral edge 420a, 420b configured to cut tissue electrosurgically in the presence of monopolar radio frequency energy from an electrosurgical unit (e.g., 10, 500). As shown in FIGS. 2 and 4A, the thickness “T” of each electrode is shown to separate sides 404a, 404b from sides 406a, 406b, while the width W of each electrode separates the medial edges 422a, 422b from the lateral edges 420a, 420b. Lateral edges 420a, 420b may further include a bevel on either or both sides thereof to provide a single or double bevel lateral edge, respectively. A bevel 418a, 418b provided on the first side 404a, 404b is shown in FIG. 4 and a second bevel on side 406a, 406b (not pictured) creates a double bevel lateral edge 420a, 420b.

Electrodes 102a, 102b further include a distal or distal-most end or tips 402a, 402b which may comprise a U-shaped portion. Lateral edges 420a, 420b, as well as distal ends 402a, 402b may be configured to cut tissue electrosurgically in the presence of monopolar radio frequency energy from electrosurgical unit 10, 500 with our without fluid 12 being provided from a fluid source 20. Further, in some embodiments, electrodes 102a, 102b and in particular lateral cutting edges 420a, 420b may be configured to cut tissue mechanically without electrosurgical energy. Further still, while two cutting edges 420a, 420b are shown, only one of the edges 420a, 420b may be configured to cut tissue electrosurgically or mechanically.

Medial edges 422a, 422b are also shown as including a bevel at least on a first side 404a, 404b (with a second bevel not shown on 406a, 406b) to provide a double bevel medial edge. However, unlike double bevel lateral edges 420a, 420b, double bevel medial edges 422a, 422b may not be intended to cut tissue and may exist predominately as a result of electrodes 102a, 102b being interchangeable for ease of manufacturing.

With continued reference to FIGS. 4A and 4B, distal ends 402a, 402b of electrodes 102a, 102b are rounded from each medial edge 422a, 422b to the lateral edge 420a, 420b and each rounded distal end 402a, 402b may be defined by a uniform radius. In this manner, the distal end of the device is void of sharp corners which could inadvertently snag, pierce or otherwise damage tissue. Each electrode 102a, 102b may further include a distal portion 408a, 408b which is at an obtuse angle A relative to side 420a, 420b and may have the general shape of a ski tip and further may be malleable such as described in the '515 Application. Likewise as described in the '515 Application, electrodes 102a, 102b may include a protrusion 410a, 410b located on side 406a, 406b. Each protrusion 410a, 410b may comprise a convex curvature, shaped like a circular dimple, which provides a blunt, rounded shape which in turn provides a smooth contour surface. In this embodiment, the convex curvature has a diameter in the range of and any increment between 0.5 mm to 1.5 mm, and more specifically in the rang of an any increment between 0.75 mm to 1.15 mm. Protrusion 410a, 410b may be formed by stamping or otherwise forming a recess 436a, 436b or concave curvature in distal portion 408a, 408b on side 404a, 404b using a stamping die or other forming die.

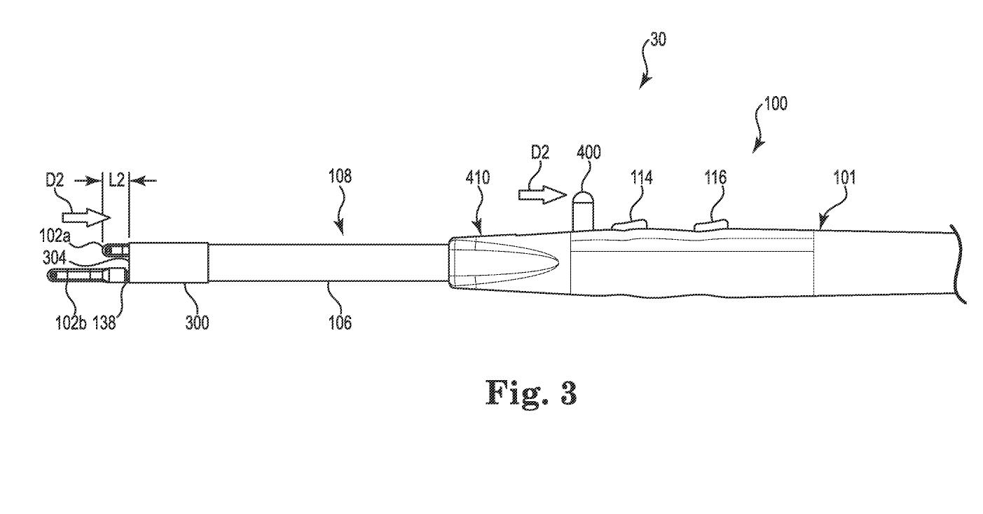

Electrode protrusions 410a, 410b may be used to move and slide electrodes 102a, 102b with painting action across a tissue surface in the presence of bipolar radio frequency energy from electrosurgical unit 10, 500 and fluid 12 from the fluid source 20, while at the same time functioning as standoffs to separate lateral edges 420a, 420b from contacting the tissue surface and inhibit edges 420a, 420b from cutting the tissue when the device 30 is used in this orientation. In this orientation, sides 404a, 404b may be referred to as the upper sides relative to the tissue being treated while sides 406a, 406b may be referred to as the lower sides. In order to best facilitate use of the handpiece 100 (FIG. 3) in the foregoing manner, electrodes 102a, 102b are shown to be coplanar (i.e., a thickness of each electrode 102a, 102b is in a common plane).

As best shown in FIGS. 4A and 4B, electrodes 102a, 102b comprise sleeve portions 412a, 412b. Sleeve portions 412a, 412b attach electrodes 102a, 102b to fluid delivery shafts 103a, 103b via cavities or passages 414a, 414b sized to receive shafts 103a, 103b, respectively. Electrodes 102a, 102b and particularly sleeve portions 412a, 412b may be crimped, pressed or welded to shafts 103a, 103b. Coupled in this manner, shafts 134a, 134b may be concentrically mated with electrodes 102a, 102b and thus fluid outlets 136a, 136b are concentrically aligned with electrode sleeve portions 412a, 412b. Upon coupling of electrodes 102a, 102b to shafts 103a, 103b, electrodes 102a, 102b extend distally from shafts 103a, 103b and fluid from shaft lumens 134a, 134b can be expelled from fluid outlets 136a, 136b located at the distal end of shafts 103a, 103b and adjacent the electrodes 102a, 102b on the same side as protrusions 410a, 410b. Fluid can exit lumens 134a, 134b through outlets 136a, 136b and travel along a groove 440a, 440b on the underside 406a, 406b of each electrode 102a, 102b and may further flow adjacent electrodes 102a, 102b such as known in the art. Grooves 440a, 440b may create a corresponding protrusion 442a, 442b on the top side 404a, 404b of each electrode.

In alternative embodiments, sleeve portions 412a, 412b may be arranged such that when electrodes 102a, 102b are coupled to shafts 103a, 103b, fluid outlets 136a, 136b are located on the opposite side of protrusions 410a, 410b. In this manner, fluid from fluid outlets 136a, 136b may flow distally on surfaces 404a, 404b to recesses 436a, 436b. Thereafter, once recesses 436a, 436b are filled with fluid, the fluid may overflow the recesses 436a, 436b and flow out of the recesses to protrusions 410a, 410b.

In still further alternative embodiments, the sleeve portions 412a, 412b do not create a full circumferential member and electrodes 102a, 102b are attached to the distal ends of fluid delivery shafts 103a, 103b via a tab, semi-circular member or other connecting member located at the proximal ends of the electrodes 102a, 102b.

In some embodiments, one or both shafts 103a, 130b may be made of electrically non-conducting material except for the portion at the distal end that comes in contact with electrodes 102a, 102b. In these embodiments, an insulated wire conductor would extend and be joined to the electrically conducting portion of shaft 103a, 103b. In still other embodiments, shafts 103a, 103b may completely comprise electrically non-conducting material, in which case an insulated wire conductor would extend and be joined directly to electrodes 102a, 102b.

Regardless of the particular shape of electrodes 102a, 102b, one of the electrodes comprises a retractable electrode as further described herein below. In the embodiments shown, reference is made to the first or upper electrode 102a as comprising a retractable electrode. However, in various other embodiments, it is envisioned that a lower or bottom electrode 102b may comprise the retractable electrode. Further, one of the electrodes 102a, 102b has a fixed or constant length L (FIG. 2). In the illustrated embodiments, electrode 102b has a fixed length L. In other words, electrode 102b does not extend and retract with respect to shaft 108 or shaft body 106 and in this manner is “non-retractable” or “axially fixed”.

As described above, the shaft sleeve 300 includes a cavity 304 sized to receive the retractable electrode 102a. FIG. 2 depicts both electrodes 102a, 102b in fully extended positions such that the distal-most tips or U-shaped portions 402a, 402b are fully extended with respect to the shaft 108 or shaft body 106. In the fully extended position, the distal-most tip 402a of the retractable electrode 102a may be approximately aligned with distal-most tip 402b of the non-retractable electrode 102b (i.e., the distal-most tips 402a and 402b are positioned and extend to approximately the same distance from the distal end 138 of the shaft 108 or shaft body 106) such as clearly shown in FIG. 2. Further, in the fully extended position, the electrodes 102a, 102b may be said to be substantially coplanar. Electrode 102a, when in a fully extended position, and electrode 102b, may have an overall (exposed) length L. Each electrode 102a, 102b may have a width W. Thus, when both electrodes 102a, 102b are extended, taking into account the space GS between the electrodes 102a, 102b, the electrodes may collectively define a first working electrode width W2. Width W2 extends from lateral edge 420a to lateral edge 420b. However, when electrode 102a is retracted into sleeve 300, the working electrode width is significantly decreased to a second working electrode width comprising only the width W of the exposed, fixed length electrode 102b. In other words, when electrode 102a is retracted, the working electrode width is decreased to a range equal to the width W of the electrode 102b. This decrease in working electrode width presents a key advantage to a user of the device. With the decrease in the electrode width (via retraction of electrode 102a), a user is able to position the single electrode 102b into a smaller or tighter space than possible when both electrodes 102a, 102b are fully exposed.

When retractable electrode 102a is in a retracted position, the overall exposed length L2 of the electrode 102a may be any incremental length less than length L (i.e., less than the fully extended length, L). In other words, the retractable electrode 102a may partially retract into cavity 304 such that various incremental lengths of the retractable electrode 102a may be exposed. Further, the retractable electrode 102a may fully retract into shaft sleeve cavity 304 such that no portion of the retractable electrode 102a is exposed or visible beyond the distal end 138 of the shaft 108 or shaft body 106. Stated otherwise, the distal-most portion 402a of retractable electrode 102a may retract into the shaft sleeve cavity 304 such that no portion of the retractable electrode 102a is exposed or viewable by a user.

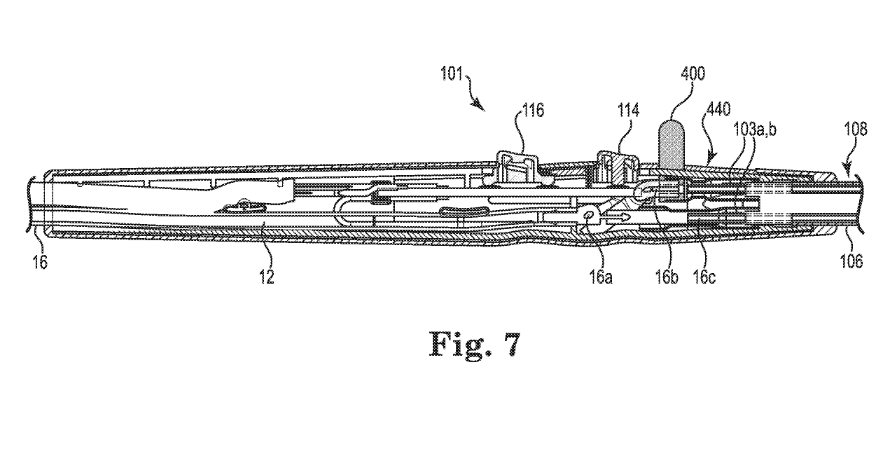

To selectively extend and retract the retractable electrode 102a, a knob 400 provided in or on handle 101 is selectively moved (i.e., pushed or pulled) distally or proximally. As shown in FIG. 7, knob 400 is coupled to shaft 103a, which, as described above, is in turn coupled to the retractable electrode 102a. Therefore, actuation of knob 400 moves the retractable electrode 102a distally or proximally depending upon the direction of movement of the knob 400. Arrows D1 (FIG. 2) illustrate the direction in which knob 400 is moved or pushed (e.g., via a finger of a user) in order to extend or move electrode 102a in distal direction (i.e., distally). Knob 400 is allowed to travel in a track 440 (as best illustrated in FIG. 7) provided on the handle 101. Arrows D2 (FIGS. 3-5) illustrate the direction in which knob 400 is moved or pushed in order to retract or move retractable electrode 102a in a proximal direction (i.e., proximally).

FIG. 7 depicts knob 400 in a fully retracted position such that the retractable electrode 102a (not shown in FIG. 7) would thereby be retracted, at least partially, into cavity 304 of shaft sleeve 300. In some embodiments the device 30 can be configured such that when the knob 400 is fully retracted in track 440, at least a portion of the retractable electrode 102a is exposed or visible such as depicted in FIGS. 3 and 5. Alternatively, in some embodiments, the device 30 and particularly the knob 400 may be configured such that when the knob 400 is fully retracted in track 440, no portion of the retractable electrode 102a is exposed or visible. In yet further embodiments according to the disclosure, the device 30 and in particular the knob 400 can be configured such that a stop or lock (not shown) engages the knob 400 to secure the retractable electrode 102a in a desired position within cavity 304 of the shaft sleeve 300. The particular exposed length L2 of retractable electrode 102a may be selected to allow for an acceptable field of view (i.e., to provide for a less obstructed or unobstructed view of the second electrode 102b) for a user of the device 30 when configuring the device 30 for use in a monopolar mode. Further, the particular exposed length L2 of retractable electrode 102a may be selected to allow for positioning of electrode 102b into a smaller or tighter space than would be possible with electrode 102a exposed or partially exposed.

As shown in FIGS. 6 and 7, device 30 includes electrical cord 40 which is connectable to electrosurgical unit 500 via cartridge 518 to selectively provide device 30 with bipolar and monopolar power output, respectively, from electrosurgical unit 500. Therefore, when the device 30 is desired to be configured for use in a bipolar mode, for example to provide for coagulation, hemostasis or sealing of tissue, a user may extend or secure the retractable electrode 102a in the fully extended position, connect the electrodes 102a, 102b to a source of bipolar energy, and activate the bipolar energy. Furthermore, when the device 30 is desired to be configured for use in a monopolar mode, for example to provide cutting or dissecting of tissue, a user may at least partially retract the retractable electrode 102a into cavity 304, connect the second electrode 102b to a source of monopolar energy and activate the monopolar energy as is known in the art. When configured for use in a monopolar mode, the retractable electrode 102a may be inactive or not connected to a source of energy.

With continued reference to FIGS. 6 and 7, during use of the device 30, fluid 12 from a fluid source 20 may be communicated within the device 30 through a tubular fluid passage provided by various structures. In the embodiment shown, fluid 12 from the fluid source 20 may be spliced for example at a fluid connection zone 16a and may be provided to two different fluid branches 16c and 16b in device handle 101. Fluid branches 16b, 16c are fluidly coupled to tubular passages or lumens 134a, 134b of electrically conductive shafts 103a and 103b respectively. Fluid 12 may flow out of fluid outlets 136a, 136b at a distal end of the device 30 as described above. Fluid 12 may comprise a liquid saline solution or another electrically conductive material. Further, an electrically non-conductive fluid may also be used with embodiments of the present disclosure. Regardless, providing fluid simultaneously with energy delivery may advantageously reduce occurrence of tissue sticking to the electrode or electrodes 102a, 102b of the device 30.

As described above, the device 30 may be used as a monopolar or a bipolar device. In this manner, the device 30 may be said to be a multipurpose device 30. As described, multipurpose device 30 may be selectively configured for use in a monopolar and a bipolar mode. As further described above, configuring the device for use in a monopolar mode may include selectively retracting the retractable electrode 102a into cavity 304 of sleeve 300. Retracting the retractable electrode 102a at least partially into cavity 304 of sleeve 300 may provide for a less obstructed field of view for a user such that the non-retractable electrode 102b may be utilized as a monopolar electrode in a more precise, bidirectional manner. In other words, when the retractable electrode 102a is retracted into the sleeve 300, since electrode 102a is no longer within the field of view (i.e., is no longer exposed to provide the collective working electrode width W2), tissue may be cut bi-directionally. That is, a user of the device 30 may cut tissue distally/proximally by moving the device 30 in a forward or backward motion using the same hand (i.e,. left-handed or right-handed) by merely changing direction of wrist or hand motion without changing the orientation of the device or blades.

Although the present disclosure has been described with reference to preferred embodiments, workers skilled in the art will recognize that changes can be made in form and detail without departing from the spirit and scope of the present disclosure.

What is claimed is:

1. A multipurpose electrosurgical device comprising:
a handle;
a shaft distal to the handle;
a first electrode and a second electrode adjacent a distal end of the shaft;
wherein the first electrode and second electrodes are laterally and spatially separated, comprise one of a same size and a same shape and each have a blunt, rounded distal end;
wherein the first electrode is an upper, retractable electrode that is movable relative to the shaft and is configured to retract into a cavity of a sleeve adjacent the distal end of the shaft; and
wherein the first electrode is coupled to a first fluid delivery shaft having a first fluid delivery lumen and a first fluid outlet and the second electrode is coupled to a second fluid delivery shaft having a second fluid delivery lumen and a second fluid outlet.

2. The device of claim 1 further comprising:
a knob coupled to the first shaft for selectively retracting and extending the retractable electrode to selectively position the retractable electrode in a retracted position wherein at least a portion of the retractable electrode is positioned in the cavity and a fully extended position wherein no portion of the retractable electrode is positioned in the cavity.

3. The device of claim 2 wherein the knob is provided in a track on the handle and is configured to slide within the track.

4. The device of claim 2 wherein, in the retracted position, the retractable electrode extends beyond a distal end of the shaft such that the retractable electrode is partially exposed.

5. The device of claim 2 wherein, in the retracted position, the retractable electrode fully retracts into the shaft sleeve cavity such that no portion of the retractable electrode is exposed.

6. The device of claim 2, wherein when the retractable electrode is in a retracted position, the device is configured for use in a monopolar mode.

7. The device of claim 2, wherein when the retractable electrode is in the fully extended position, the device is configured for use in a bipolar mode.

8. The device of claim 2, wherein the first and second electrodes each comprise a width; and wherein a spatial gap separation is provided between the first and second electrodes such that when the retractable electrode is in the fully extended position, the first and second electrodes in combination with the spatial gap separation comprise a collective working electrode width and when the retractable electrode is in a retracted position, a working electrode width comprises only the width of the second electrode.

9. The device of claim 1, wherein the first and second electrodes further comprise first and second sleeve portions comprising first and second sleeve passages and wherein a distal end of the first fluid delivery shaft is received within the first sleeve passage and a distal end of the second fluid delivery shaft is received within the second sleeve passage to thereby couple the first and second fluid delivery shafts to the first and second electrodes.

10. The device of claim 9, wherein the first fluid outlet is concentrically aligned with the first sleeve portion and the second fluid outlet is concentrically aligned with the second sleeve portion.

11. The device of claim 1, wherein the first fluid delivery shaft is coupled to a first electrical conductor and the second fluid delivery shaft is coupled to a second electrical conductor and wherein the first and second electrical conductors are selectively connectable to a bipolar power output source and the second electrical conductor is selectively connectable to a monopolar power output source.

12. The device of claim 1, wherein at least the second electrode provides a lateral cutting edge and is configured for bidirectional cutting of tissue when the first electrode is in a refracted position.

13. The device of claim 1, wherein the first and second electrodes comprise a ski tip shape.

14. A method of selectively applying monopolar and bipolar energy to tissue during an electrosurgical procedure with a single electrosurgical device having a handle comprising a shaft, the method comprising:
configuring the device to a bipolar mode comprising:
extending a retractable electrode from a shaft sleeve cavity comprising moving the retractable electrode distally relative to the shaft such that the retractable electrode is in a fully extended position and a distal end of the retractable electrode is approximately aligned with an adjacent, non-retractable second electrode; and
applying bipolar energy to the tissue via the first and second electrodes;
configuring the device to a monopolar mode comprising:
retracting the retractable electrode into the shaft sleeve cavity comprising moving the retractable electrode proximally relative to the shaft such that the retractable electrode is in a retracted position; and
applying monopolar energy to the tissue via the second electrode;
wherein the first electrode is coupled to a first fluid delivery shaft having a first fluid delivery lumen and the second electrode is coupled to a second fluid delivery shaft having a second fluid delivery lumen; and
wherein the first electrode and second electrodes are laterally and spatially separated, comprise one of a same size and a same shape and each have a blunt, rounded distal end.

15. A multipurpose electrosurgical device selectively configurable for use in a monopolar and a bipolar mode, the device comprising:
a handle;
a shaft distal to the handle;
a first electrode tip having a first sleeve portion and a second electrode tip having a second sleeve portion, the first electrode tip spaced from the second electrode tip, the first and second electrode tips adjacent a distal end of the shaft;
wherein the first electrode tip comprises a retractable electrode configured to at least partially retract into a cavity in the shaft and the second electrode tip comprises an axially fixed electrode tip; and
wherein the first sleeve portion is coupled to a first fluid delivery shaft having a first fluid outlet and the second sleeve portion is coupled to a second fluid delivery shaft having a second fluid outlet such that the first and second fluid delivery outlets are concentrically aligned with the first and second sleeve portions respectively.

16. The multipurpose electrosurgical device of claim 15, wherein when the retractable electrode is at least partially retracted into a cavity in the shaft, the retractable electrode tip is partially exposed and the device is configured for use in a monopolar mode.

17. The multipurpose electrosurgical device of claim 15, wherein the retractable electrode tip comprises a fully extended position and wherein when the retractable electrode tip is in the fully extended position, the retractable electrode tip is completely exposed and the device is configured for use in the bipolar mode.
